(19) United States
(12) Patent Application Publication
(10) Pub. No.: US 2014/0288539 A1
(43) Pub. Date: Sep. 25, 2014

(54) Ophthalmic Laser System and Method for Severing Eye Tissue

(71) Applicant: Carl Zeiss Meditec AG, Jena (DE)

(72) Inventors: Mark Bischoff, Jena (DE); Gregor Stobrawa, Jena (DE); Elke Ebert, Jena (DE)

(73) Assignee: Carl Zeiss Meditec AG, Jena (DE)

(21) Appl. No.: 14/352,696

(22) PCT Filed: Oct. 18, 2012
(86) PCT No.: PCT/EP2012/070630

§ 371 Date: Apr. 18, 2014

(30) Foreign Application Priority Data
Oct. 20, 2011 (DE) 10 2011 116 759.9

(51) Int. Cl. A61F 9/008

(52) U.S. Cl. CPC A61F 9/00836; A61F 2009/00872; A61F 2009/00897; A61F 2009/00882
USPC 606/4

(57) ABSTRACT

An ophthalmic laser device with a treatment beam path that includes a variably adjustable modulator. The ophthalmic laser device is adapted to determine for the radiation pulses to be inputted into the tissue a power density to be inputted into the relevant target volume through the treatment beam path depending on a spatial distance between two target volumes of immediately successive radiation pulses and to adjust the modulator parallel in time with the control of the deflecting unit such that the relevant pulse in the target volume has the determined power density.

The present application claims priority from PCT Patent Application No. PCT/EP2012/070630 filed on Oct. 18, 2012, which claims priority from German Patent Application No. DE 10 2011 116 759.9 filed on Oct. 29, 2011, the disclosures of which are incorporated herein by reference in their entirety.

### FIELD OF THE INVENTION

The invention is directed to an ophthalmic laser device for severing eye tissue by optically generated, nonlinear interaction, with an ultrashort pulse laser having radiation pulses which can be focused in different target volumes along a treatment beam path which comprises a variably adjustable beam deflecting unit (for scanning the eye tissue) and focusing optics by means of the deflecting unit, and a control unit for controlling the laser and for controlling the deflecting unit during the emission of a sequence of radiation pulses, and to a method for severing eye tissue, particularly cornea, by means of at least one laser incision through input of energy by means of a sequence of radiation pulses focused in target volumes, particularly on tracks along (two-dimensionally) curved spatial areas, particularly along contour lines of such spatial areas. The laser device serves in particular to carry out a method of this type. Within the meaning of the invention, an ultrashort pulse laser is a laser which is capable of emitting pulses with a duration in the femtosecond range or picosecond range.

Within the meaning of the invention, an individual laser incision for severing eye tissue comprises a field of two-dimensionally distributed interaction zones in which the input energy alters the tissue, particularly on a molecular level. This field is generated by scanning the tissue to be severed by means of the deflecting unit during the emission of radiation pulses. The energy of every radiation pulse is inputted with a power density into a relevant target volume in which the radiation pulse is focused. The input energy may partially reach the surrounding tissue through transport processes such that an interaction zone may be larger than the irradiated target zone. In many cases, but not always, gas bubbles are formed in the course of the nonlinear interaction between laser radiation and material in the interaction zone and, therefore, a field of two-dimensionally distributed gas bubbles is brought about.

It is noted that citation or identification of any document in this application is not an admission that such document is available as prior art to the present invention.

Laser devices and methods of this type are known in the art, for example, from WO 2005/011547 A1, which disclosure is incorporated in its entirety herein. It describes the generation of two-dimensionally curved laser incisions. The interaction zone fields are generated in that the radiation pulses are inputted at least partially in target volumes located along circular or spiral tracks. The circular or spiral tracks form contour lines of two-dimensionally curved surfaces which describe the shape of the incision to be made.

It is known from WO 2007/042190 A2, which disclosure is incorporated in its entirety herein, that interaction zones in which no optical breakdown is generated by radiation pulses of lower power densities and which are correspondingly small are also suitable for severing eye tissue when the irradiated target volumes are located close enough together or even overlap one another. This applies to the spatial distance between target volumes into which immediately successive radiation pulses are inputted (spot distance) as well as to the spatial distance between adjacent target volumes of different track portions (track distance).

It is advantageous for both patient and physician if the process of laser-assisted severing of eye tissue can be shortened. However, in case of laser incisions in the eye (cornea, lens, etc.), shortening of the process is confronted by technical limitations. For laser devices with field objective, the process can indeed be shortened, for example, by increasing the pulse repetition frequency (hereinafter: pulse frequency). However, a higher pulse frequency heightens requirements particularly for the beam deflecting system. For this reason, in the prior art the pulse frequency is reduced again for a portion of the irradiated area of the eye tissue. The reduction serves in particular to produce at least approximately constant spot distances and at least approximately constant track distances. This serves to prevent the formation of very large gas bubbles or other kinds of irregularities when making incisions which could lead to clinical complications.

It is known from WO 2005/058216 A1 to reduce the mean (effective) pulse frequency by means of a fast optical switch. This is accomplished in that some of the radiation pulses are selected from the sequence of radiation pulses with adjustable frequency and attenuated or otherwise modified. Only the rest of the pulses (those not selected) bring about the interaction effect desirable for severing, typically an optical breakdown (photodisruption). The fast optical switches required for this purpose are costly and are fundamentally limited in technical respects regarding their maximum switching frequency. Therefore, they represent a limiting element for further shortening of the process for very high pulse frequencies between 1 MHz and approximately 10 MHz combined with pulse energies of greater than 10 nJ.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

It is further noted that the invention does not intend to encompass within the scope of the invention any previously disclosed product, process of making the product or method of using the product, which meets the written description and enablement requirements of the USPTO (35 U.S.C. 112, first paragraph) or the EPO (Article 83 of the EPC), such that applicant(s) reserve the right to disclaim, and hereby disclose a disclaimer of, any previously described product, method of making the product, or process of using the product.

### SUMMARY OF THE INVENTION

The object of the invention is to provide an ophthalmic laser device and a method of the type mentioned above which allow higher pulse frequencies to be used in order, for example, to shorten the treatment process.

According to the invention, it is provided that the treatment beam path of the ophthalmic laser device comprises a variably adjustable modulator and that the control unit is adapted to determine for a radiation pulse, or for every radiation pulse of a sequence, a power density to be inputted into the relevant target volume through the treatment beam path depending on a spatial distance between two target volumes of immediately successive radiation pulses and to adjust the modulator to the (respective) determined energy in parallel with the control of the deflecting unit. As spatial distance for determining the power density, the control unit preferably utilizes the spot distance between the relevant target volume and the target volume associated either with the immediately preceding radiation pulse or with the immediately succeeding radiation pulse or utilizes the spot distance of the two target volumes which are to be generated (or which were already generated) immediately preceding the relevant target volume. Alternatively, the control unit can use a mean spot distance in a region of predetermined shape and size around the relevant target volume as spatial distance. The determination of the power density can be carried out temporally and/or spatially independently of the adjustment of the power density by means of the modulator; for example, it can be carried out before the start of the emission of the radiation pulses. In particular, the power density can be stored in the system as known function in the form of a table or as a mathematical formula.

The modulator can influence one or more physical parameters of each radiation pulse of the laser passing it such that the power density of the pulse in the relevant target volume is defined thereby. In particular, useful physical parameters include the phase, amplitude, intensity, polarization, beam direction (Poynting vector) or the field distribution over the beam cross section (beam profile). In particular, these parameters can also be manipulated in the frequency domain, for example, in spatial-spectral splitting, because this is made possible in a simpler manner when changing ultrashort pulses. In particular, the influencing can vary at different wavelengths of the same pulse. The modulator can comprise a plurality of elements, also spaced-apart elements, for example, an adjustable polarizer and a fixed polarizer (analyzer). In the simplest case, the modulator is an energy modulator which is able to reduce the energy of the radiation pulses.

A tissue-damaging effect of target volumes which are arranged too close together and are therefore irradiated too intensively can be prevented in an economical manner on the one hand and a uniformly good severing of tissue can be achieved on the other hand in that the power density to be inputted into the respective target volume is controlled on the basis of individual pulses as a function of the spot distances to be generated (or which have already been generated) in these target volumes. In contrast to the prior art, irradiation can take place with constant (effective) pulse frequency. Accordingly, a costly fast optical switch can be dispensed with in particular. Even when an optical switch is arranged in the treatment beam path, it can be left unused so that high pulse frequencies of 1 MHz or more are possible. The treatment process can be significantly shortened in this way.

Instead of determining and adjusting the power density, any other physical quantity which describes a magnitude of the interaction of a radiation pulse with the tissue in the target volume in question and which depends on the local spot distance (and optionally on the local track distance) can be used. A quantity of this type can be used wherever power density is referred to in the preceding and hereinafter. Thus the energy density in particular can be used instead of the power density—for example, when pulse duration is (at least approximately) constant throughout the sequence of radiation pulses. Alternatively, the power can be used in place of the power density—for example, when all target volumes are (at least approximately) equally focused and if the beam shape is (at least approximately) constant for all radiation pulses. Alternatively, the energy can be used instead of the power density—for example, when the pulse duration is (at least approximately) constant in addition to the target volume and beam shape.

The power density to be inputted preferably depends on the spatial distance in a mathematically monotonically increasing manner. Thus as the spot distance increases, a greater power density is inputted into the relevant target volume. In this way, in an economical manner power density can be inputted to a sufficient degree to overcome the severing threshold on the one hand and tissue damage due to excessive input of energy in case of small spot distances can be avoided on the other hand. This allows more accurate severing.

In especially preferred embodiments, the control unit controls the deflecting unit during emission such that an instantaneous focus of the radiation pulses moves along a closed track, or at least approximately closed track, particularly along a circular track, an approximately circular track or elliptical track or spiral track. A track is approximately closed, for example, when two directly adjacent track portions are located less than 10 μm apart. Tracks of this kind are described in WO 2005/011547 A1, for example. They allow two-dimensionally curved laser incisions to be made such that it is possible to use a physiologically shaped contact lens without massive deformation of the eye tissue.

The control unit preferably determines the power density to be inputted for the radiation pulse additionally as a function of a spatial distance between two directly adjacent track portions (comprising, for example, two consecutive cycles of the track movement) or directly adjacent tracks, particularly in mathematically monotonically increasing dependency of the power density to be inputted upon the spatial distance. A larger power density is then inputted into the relevant target volume as track distance increases. In this way, in an economical manner, power density can be inputted to a sufficient degree to overcome the severing threshold on the one hand and tissue damage due to excessive input of energy in case of small spot distances can be avoided on the other hand. This allows more accurate severing.

In particularly preferred embodiments, the control unit determines the power density to be inputted at each value of the spatial distance (particularly at all values, both of the spot distance and of the track distance) greater than a required power density at the relevant value of the spatial distance (particularly of the spot distance and of the track distance) for a photodisruption with predetermined probability, particularly by a constant amount which is not dependent upon the relevant value of the spatial distance (especially of the spot distance and of the track distance), particularly according to Eᵢ=E_{TH}(dₛ,dₜ)+C. The function E_{TH}(dₛ,dₜ) specifies the power density which is to be inputted into a target volume to bring about an optical breakdown with predetermined probability P_{TH}, for example 95%. There is a different function E_{TH}(dₛ,dₜ)=E_{TH}(dₛ,dₜ, P_{TH}) for each probability P_{TH}.

The constant amount is preferably between C=1 nJ and C=1 μJ.

In particularly advantageous embodiment forms, the control unit determines the power density to be inputted at each value of the spatial distance above a predetermined limit distance dₛ₀ or dₜ₀ greater than a required power density at the relevant value of the spatial distance for a photodisruption with predetermined probability and below the predetermined limit distance less than a required power density at the relevant value of the spatial distance for a photodisruption with the predetermined probability, particularly according to Eᵢ=E_{TH}(dₛ,dₜ)+E_{subTH}(dₛ,dₜ), where E_{subTH}(dₛ,dₜ)<0 for dₛ<dₛ₀ and for dₜ<dₜ₀. The distance-dependent monotonically increasing curve of Eᵢ intersects the curve of the distance-dependent threshold value E_{TH} for an optical breakdown (at given breakdown probability P_{TH}). In this way, corresponding to WO 2007/042190 A2, two regions with significantly different interaction strengths can be generated with little effort. If gas bubbles occur at the inputted power densities, these gas bubbles have correspondingly different gas bubble sizes. However, it can just as easily also be the case that gas bubbles occur only in one zone and the severing takes place in the second zone without the occurrence of gas bubbles.

Correspondingly, the invention includes an embodiment form in which the control unit controls the deflecting unit in such a way that target volumes with spatial distances above the limit distances result in an outer annular region of an interaction zone field and target volumes with spatial distances below the limit distances result in an inner region of the interaction zone field. The limit of the two regions is characterized by the limit distance (spot distance or track distance) or limit distances (spot distance or track distance). In the region in which the spot distance (optionally also the track distance) is less than the limit distance, smaller gas bubbles, or no gas bubbles, are generated because of the lower power density input, which allows a finer severing. In the other region of larger distances, larger gas bubbles are possibly generated by optical breakdowns, which brings about a coarser severing of the tissue which is visually perceptible by the person administering the treatment.

The limit distance (or limit distances) can be selected in such a way, for example, that it corresponds to a predetermined distance from the photopic pupil. Finer severing is then carried out in the optical zone region of the patient that is critical for distance vision so as to enable high visual acuity, whereas coarser severing is carried out outside this optical zone region in order to shorten the process and make the severing visually perceptible for an observer, for example, the person administering treatment.

The function E_{subTH}(dₛ,dₜ) preferably depends on the product of dₛ×dₜ of the spatial distances which gives the surface area of the elementary cell of the interaction zone field, or it is a linear function or a step function. This enables finer severing of the tissue economically with high accuracy.

The control unit can advantageously control the laser during the emission of the radiation pulses such that an emission frequency of the laser is constant, particularly at an emission frequency between 1 MHz and 10 MHz. Accordingly, costly measures for adjusting the frequency of the laser can be dispensed with. Nevertheless, the treatment process can be shortened by means of the high pulse frequency. Pulse frequencies in the above-mentioned region can be provided by means of a cavity-dumped fs oscillator, as it is called, corresponding, for example, to WO 2003/59563 A2, which disclosure is incorporated herein in its entirety. Femtosecond fiber-optic lasers in the infrared spectral range or pulsed lasers emitting in the ultraviolet spectral range are likewise suitable.

The spatial distances between directly successive target volumes (spot distances, see above) are preferably between 0.5 μm and 5 μm, particularly with spatial distances of adjacent track portions of different track cycles (track distances, see above) between 0.5 μm and 5 μm.

In the device according to the invention and method according to the invention, the spot distances and track distances need not be constant, not even approximately. However, it is possible to maintain them constant, or at least approximately constant, by adapting the scanning speed, i.e., by adapting the rate of change of the deflection by the deflecting unit. It is also possible to use constant distances of this type only in a partial area of an interaction zone field.

It is provided for the method according to the invention for severing eye tissue that with a radiation pulse (particularly with every radiation pulse of the sequence) in the relevant target volume, a power density is inputted into the relevant target volume in mathematically monotonically increasing dependency on a spatial distance between two target volumes of directly successive radiation pulses.

The method is particularly advantageous when target volumes are located on a track along a curved spatial area, particularly along a circular track, an approximately circular track or elliptical track or a spiral track, wherein the power density inputted into the relevant target volume depends additionally in a mathematically monotonically increasing manner of the spatial distance upon a spatial distance between two adjacent track portions of two different cycles of the track course.

For the rest, the method can be realized according to the above-mentioned steps carried out by the control unit.

The invention also includes a computer program which is adapted for implementation of a method according to the invention, particularly a data carrier containing a computer program of this kind.

A computer program of this kind can have one or more software modules which implement the functions of the control unit. For example, a software module can instruct the CPU of a computer to determine the power density to be inputted as a function of the local spot distance (and optionally also of the local track distance), whereas another software module controls or adjusts the modulator (later or parallel in time) according to the determined power density values.

### BRIEF DESCRIPTION OF THE DRAWINGS

FIG. 1 shows a first laser device;

FIG. 2 shows curves of threshold values for optical breakdowns;

FIG. 3 shows three functions for determining the power density to be inputted;

FIG. 4 shows a relationship between track radius and spot distance;

FIG. 5 shows a flowchart for a method according to the invention; and

FIG. 6 shows treatment results.

Corresponding parts are designated by the same reference numerals in all of the drawings.

### DETAILED DESCRIPTION OF EMBODIMENTS

It is to be understood that the figures and descriptions of the present invention have been simplified to illustrate elements that are relevant for a clear understanding of the present invention, while eliminating, for purposes of clarity, many other elements which are conventional in this art. Those of ordinary skill in the art will recognize that other elements are desirable for implementing the present invention. However, because such elements are well known in the art, and because they do not facilitate a better understanding of the present invention, a discussion of such elements is not provided herein.

The present invention will now be described in detail on the basis of exemplary embodiments.

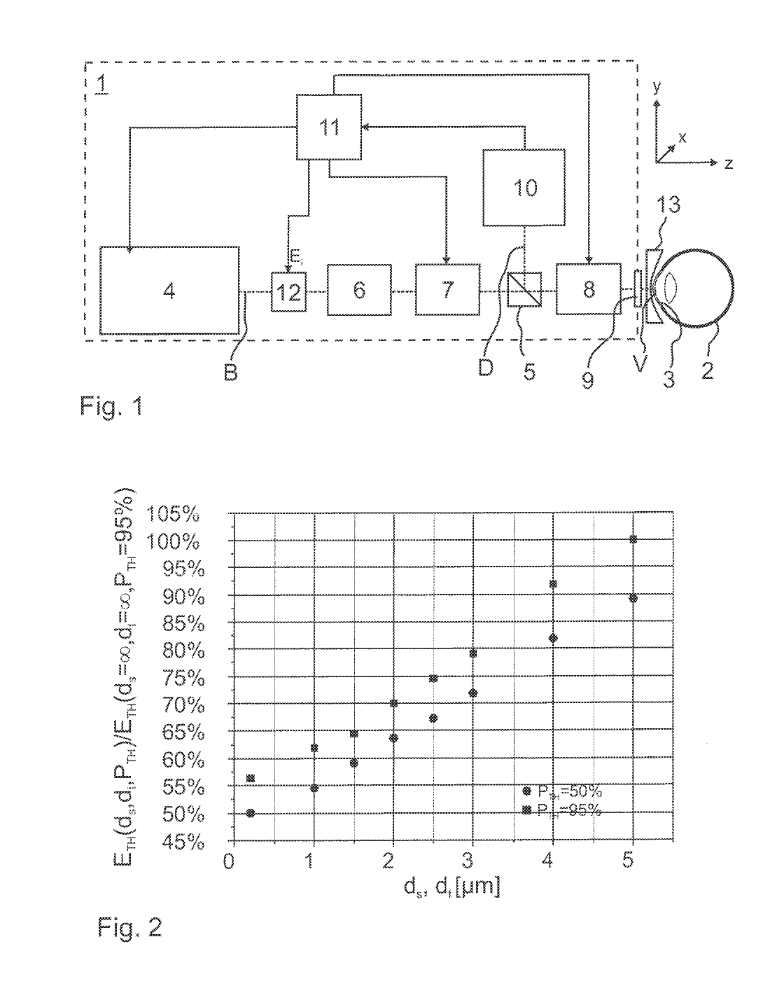

FIG. 1 shows an ophthalmic laser device 1 schematically by way of example which is provided for treatment of defective vision of an eye 2 by severing tissue of the cornea 3. It has a short pulse laser 4, for example, a pulsed TiSa infrared laser, in the treatment beam path B for emitting infrared radiation with a wavelength of 1064 nm and a pulse length between 100 fs and 1000 fs and a frequency between 1 MHz and 10 MHz. The laser device 1 further has a beamsplitter 5, scanning optics 6, an adjustable beam deflecting unit 7, adjustable focusing optics 8 and an end glass 9. The detection beam path D of a detector 10, for example, an optical coherence tomograph (OCT), is coupled out of the treatment beam path B for navigation via the beamsplitter 5. The detector 10 is connected to a control unit 11. Further, an adjustable modulator 12, for example, a combination of a polarizer which can be rotated by motor and an analyzer downstream, is arranged in the illumination beam path. The treatment beam path B accordingly extends from the laser 4 through the end glass 9 to the treatment region in which the eye 2 is to be positioned. A fixating device 13 for the eye 2 is arranged between the laser system 1 and the eye 2.

The deflecting unit 7 comprises, for example, a quantity of galvanometer mirrors for deflecting the laser beam in X direction and Y direction over the cornea 3. The focusing of the laser beam in Z direction along the optical axis is achieved, for example, by means of a movable lens or lens group within the focusing optics 8 or, alternatively, through a movable tube lens (not shown). The pulsed IR laser radiation exits the laser 4 and is focused via the scanning optics 6, the scanner unit 7 and the focusing optics 8 in an instantaneous (dependent upon the adjustment of the deflecting unit 7) target volume V in the eye lens 2. The control unit 14 can move the focus in X direction, Y direction and Z direction by means of the deflecting unit 7 and the focusing optics 8 so that a different target volume V can be irradiated.

The fixating device 13 carries out the functions of mechanically coupling the patient's eye 2 to the optical construction of the device 1, transmitting the optical beams for navigation and therapy and—optionally—enabling mechanical access options for probes or surgical instruments to the anterior chamber of the eye. The patient's eye 2 is advisably fixated prior to a detection and/or therapy, for example, is held at the fixating device 13 by suction by way of negative pressure. In addition, the head of the patient may be fixated (not shown). The patient's gaze can be kept as constant as possible by means of a suitable fixating target (not shown). In so doing, it is possible to compensate in an adjustable manner for the angle between the geometric axis and the visual axis of the eye.

The control unit 11 can determine characteristic properties of the cornea 3 such as shape, thickness and position by means of the detector 12 before treatment. Based on the characteristic properties, a mathematical model of the cornea 3 can be prepared, for example, by adapting a standardized model to the characteristic properties. Based on the model and a predefined incision geometry, the control unit 11 can determine the spatial control parameters for the deflecting unit 7 and focusing optics 8 and the energy control parameters for the modulator 12.

The control unit 11 determines the spatial control parameters in such a way, for example, that the focus of the radiation pulses moves along a track, at least a portion of which extends cyclically in at least one spatial coordinate, for example, along concentric circular tracks. The energy control parameters can indicate the respective rotational positions of the polarizer of the modulator 12 to be adjusted, for example. To this end, the modulator 12 can have its own control module which interprets the energy control parameters outputted by the control unit 11 and controls or adjusts the modulator 12 correspondingly.

Every radiation pulse emitted by the laser 4 during the subsequent treatment process is inputted into a relevant target volume Vᵢ through the treatment beam path B. The control unit 11 determines beforehand for each radiation pulse a separate energy control parameter which gives the respective power density Eᵢ to be inputted into the relevant target volume Vᵢ. The control unit determines this power density Eᵢ as a function of two spatial distances: the spot distance d, between two target volumes Vᵢ, Vᵢ₊₁ of directly successive radiation pulses on the one hand and from the track distance d, of two directly adjacent track portions from two different cycles of the track movement on the other hand. For this purpose, it uses, for example, the relationship Eᵢ=E_{TH}(dₛ,dₜ)+C, where E_{TH} indicates the power density which is to be inputted into a target volume to bring about an optical breakdown with a probability P_{TH} of 95%, and C is constantly 100 nJ, for example.

As soon as the person conducting the treatment initiates the treatment process, the control unit 11 begins to control the laser 4 to emit a sequence of radiation pulses and, temporally parallel therewith, controls the deflecting unit 7, focusing optics 8 and modulator 12 based on the determined control parameters.

FIG. 2 illustrates the function E_{TH}(dₛ,dₜ). A different function E_{TH}(dₛ,dₜ)=E_{TH}(dₛ,dₜ,P_{TH}) applies for every probability P_{TH}. The diagram shows individual values of E_{TH}(dₛ,dₜ,P_{TH}) for P_{TH}=95% and for P_{TH}=50%. For the sake of clarity, the dependencies of spot distance d, and track distance d, are presented as identical. This assumption is approximately correct when directly adjacent tracks are irradiated within a maximum 5 seconds.

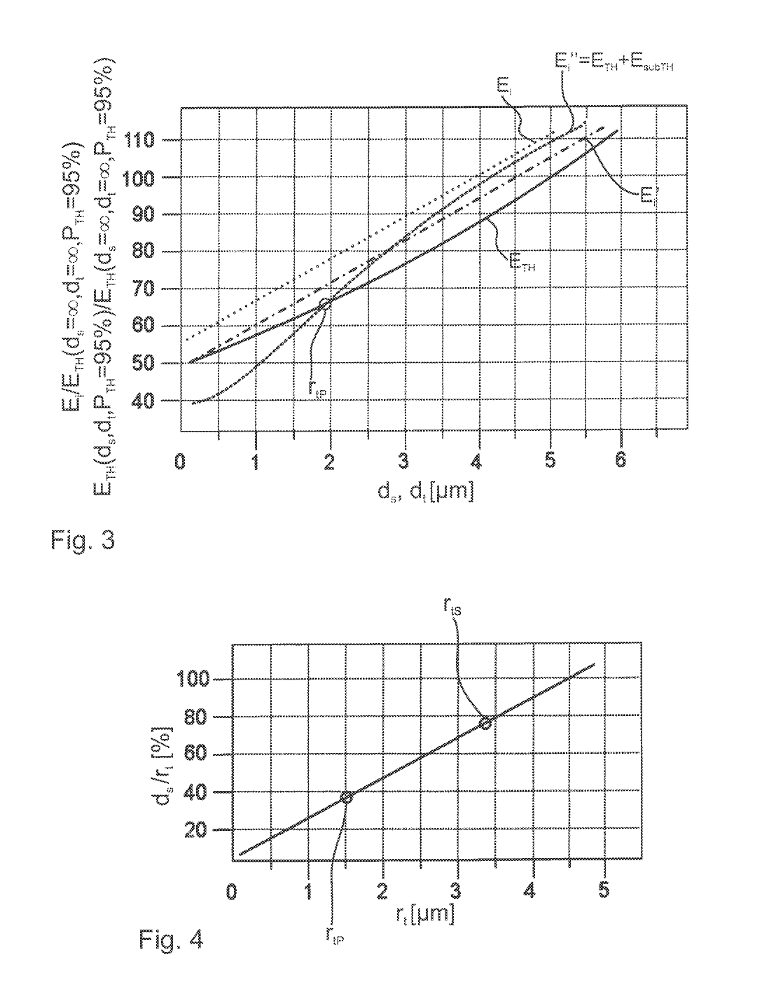

FIG. 3 shows examples for alternative relationships Eᵢ, E′ᵢ and E″ᵢ for determining the power densities to be inputted for individual pulses in addition to the function E_{TH}(dₛ,dₜ,P_{TN}=95%). All of the variants shown depend in a monotonically increasing manner on the local spot distance dₛ and on the local track distance dₜ. The dependencies of these two distances are also represented as identical here for the sake of clarity. The power densities Eᵢ and E′ᵢ are above the breakdown thresholds E_{TH} regardless of the values of the distances. The power density E″ᵢ=E_{TH}(dₛ,dₜ)+E_{subTH}(dₛ,dₜ), where E_{subTH}(dₛ,dₜ)<0 for dₛ<dₛ₀ and for dₜ<dₜ₀ intersects the breakdown threshold value at the exemplary limit distances dₛ₀=dₜ₀=1.9 μm. Accordingly, no optical breakdowns are generated below these distances but rather only gas bubbles, for example. The limit distances are selected in such a way, for example, that they correspond to a track radius r_{tP} located at the outer edge of the photopic pupil of the eye 2.

FIG. 4 schematically depicts the dependency of the spot distance d, on the track radius in a circular scanning track at constant pulse frequency. Exemplary track radius r_{tP} at the edge of the photopic pupil and track radius r_{tP}, at the edge of the scotopic pupil are also shown.

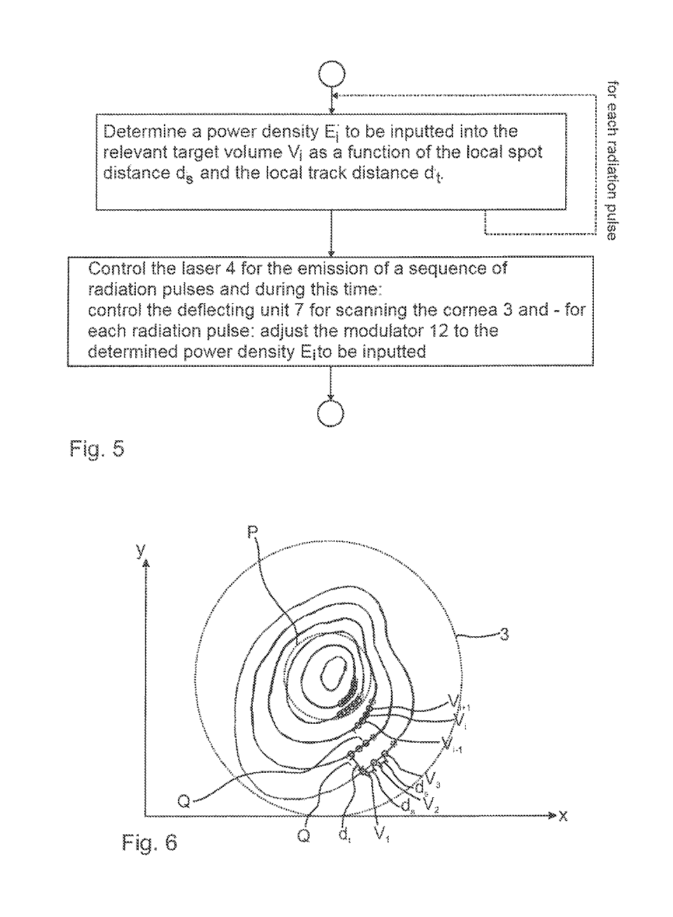

The method according to the invention is illustrated schematically in the form of a flowchart in FIG. 5. The power densities Eᵢ for all radiation pulses can be determined beforehand before starting the irradiation or during the radiation, for example, in real time before the emission of each radiation pulse.

A treatment is carried out comprising two laser incisions at different depths, for example, one of which opens a flap. Irradiation is carried out in the annular outer region of the flap with 3 μm mean (or constant) spot distance and track distance and in the inner region with spot distances and track distances between 3 μm and 0.3 μm, for example. In the inner region, there is a linear (or quadratic or root type) relationship between the spiral track radius and the spot distance or track distance. At the same time, the pulse energy in the annular outer region of an incision surface is 140 nJ and goes down to 50 nJ in the inner region. For spot distances and track distances located therebetween, a corresponding mean pulse energy is selected. The target volumes are 20 μL and the duration of the radiation pulses is 20 fs, for example.

FIG. 6 shows a result of a method according to the invention in which target volumes Vᵢ were irradiated by means of the deflecting unit 7 and the focusing optics 8 along contour lines of the cornea 3 according to the relationship to E″ᵢ from FIG. 3. The spot distances and track distances lie between 0.5 μm and 5 μm. The movement of the beam between the individual tracks was carried out at interconnections Q which were not irradiated. It will be seen that the spot distances at outer tracks (with large track radii) are larger than in the inner tracks. In the innermost tracks, the target volumes Vᵢ even overlap one another. Within the photopic pupil P, the radiated power densities E″ᵢ lie below the breakdown thresholds E_{TH} so that the laser incision is very finely formed in this case.

If the track described by the deflecting unit 7 is spiral-shaped (not shown) and the rotational frequency is always maintained at the upper technical limit of the deflecting unit 8, there results a variable track speed v(rₜ) which increases monotonically with the track radius rₜ. As a result, in connection with a constant (effective) pulse frequency, the spot distances dₛ increase monotonically with radius rₜ along the track curve. It lies within the scope of the invention that the pulse power density Eᵢ to be inputted is kept constant in that the track distance is adapted in a radius-dependent manner by corresponding control of the deflecting unit 7 such that the combination of radius-dependent spot distances and track distances dₛ(rₜ), dₜ(rₜ) leads to the same pulse power density depending on function E_{TH}(dₛ,dₜ). Accordingly, it is not necessary to adapt the pulse power density during the scanning process. To determine the power density for each radiation pulse, the control unit 11 can use the relationship Eᵢ=dₛ*dₜ*E₀, for example, which leads to Eᵢ=const. when it determines spatial control parameters for the deflecting unit 7 based on dₜ=1/dₛ, and the pulse parameters (duration, shape, etc.) remain constant in other respects.

In all of the embodiment forms, it is equivalent when determining the power densities Eᵢ(‘,”) to be inputted to use a dependency of the local track speed v instead of the dependency of the local spot distance dₛ because the relationship between spot distance and track speed is given by the pulse frequency.

Instead of the described combination of polarizer and analyzer, an acousto-optical modulator (AOM), an electro-optical modulator (EOM), a Pockels cell, a liquid crystal (LC) element, a fiber-optic switching element or a chopper wheel can be used as modulator 12 and optionally supplemented in each instance by components which bring about a transformation of the optical characteristics of the selected laser pulses primarily changed in this way such that a physical parameter defining the power density in the target volume is modified in a defined manner. To this end, for example, the laser pulse can be lengthened (stretched) with respect to time by dispersion. This effect can be achieved, for example, by an adjustable polarization rotation of the selected laser pulses by means of a suitable transformation—for example, through the use of polarization-dependent reflection. Fast polarization rotations of this type can be brought about, for example, by Pockels cells. An alteration of the wavefront of the radiation pulses leading to incomplete focusing and, therefore, to a reduction in the (peak) power density is also possible. Wavefront alterations of this kind can be achieved, for example, by liquid crystal elements or also by diaphragm mirrors such as are known from adaptive optics.

While this invention has been described in conjunction with the specific embodiments outlined above, it is evident that many alternatives, modifications, and variations will be apparent to those skilled in the art. Accordingly, the preferred embodiments of the invention as set forth above are intended to be illustrative, not limiting. Various changes may be made without departing from the spirit and scope of the inventions as defined in the following claims.

### LIST OF REFERENCE NUMERALS

1 ophthalmic laser device
2 eye
3 cornea
4 ultrashort pulse laser
5 beamsplitter
6 scanning optics
7 deflecting unit
8 focusing optics
9 end glass
10 detector
11 control unit
12 modulator
13 fixating device
B treatment beam path
D detection beam path
V(ᵢ) target volume
Eᵢ energy to be inputted
P photopic pupil
r_{tP} track radius at the outer edge of the photopic pupil

1. An ophthalmic laser device for severing eye tissue, comprising:
an ultrashort pulse laser configured to focus radiation pulses in different target volumes along a treatment beam path;
a variably adjustable beam deflecting unit;
focusing optics;
a control unit configured to control the laser and configured to control the deflecting unit during an emission of a sequence of radiation pulses; and
variably adjustable modulator;
wherein the variably adjustable beam deflecting unit, the focusing optics, and the variably adjustable modulator are arranged along the treatment beam path;
wherein the control unit is configured to determine for a radiation pulse, or for every radiation pulse of the sequence, a power density (Eᵢ) to be inputted into the relevant target volume through the treatment beam path depending on a spatial distance dₛ between two target volumes of immediately successive radiation pulses; and
wherein the control unit is configured to adjust the modulator parallel in time with the control of the deflecting unit such that the relevant pulse in the target volume has the determined power density (Eᵢ).

2. The laser device according to claim 1;
wherein the power density to be inputted depends in a mathematically monotonically increasing manner on the spatial distance dₜ.

3. The laser device according to claim 1;
wherein the control unit controls the deflecting unit during emission such that an instantaneous focus of the radiation pulses moves along a dosed track, or at least an approximately closed track.

4. The laser device according to claim 1;
wherein the control unit determines the power density (Eᵢ) to be inputted for the radiation pulse additionally as a function of a spatial distance dₜ between two directly adjacent track portions or directly adjacent tracks.

5. The laser device according to claim 1;
wherein the control unit determines the power density (Eᵢ) to be inputted at each value of the spatial distance dₛ to be greater than a required power density (E_{TH}) at the relevant value of the spatial distance dₛ for a photodisruption with predetermined probability.

6. The laser device according to claim 5;
wherein the power density (Eᵢ) is determined to be greater than the required power density (E_{TH}) by a constant amount between 1 nJ and 1 μJ.

7. The laser device (1) according to claim 1;
wherein control unit determines the power density (Eᵢ) to be inputted, for each value of the spatial distance dₛ above a predetermined limit distance, to be greater than a required power density (E_{TH}) at the relevant value of the spatial distance for a photodisruption with predetermined probability; and
wherein control unit determines the power density (Eᵢ) to be inputted, for each value of the spatial distance dₛ below the predetermined limit distance, to be less than the required power density (E_{TH}) at the relevant value of the spatial distance for a photodisruption with the predetermined probability.

8. The laser device according to claim 4;
wherein control unit determines the power density (Eᵢ) to be inputted, for each combination of the spatial distance dₛ and the special distance dₜ above a predetermined limit distance, to be greater than a required power density (E_{TH}) at the relevant value of the combination of spatial distances dₛ and dₜ for a photodisruption with predetermined probability:
wherein control unit determines the power density (Eᵢ) to be inputted, for each combination of the spatial distances dₛ and dₜ below the predetermined limit distance, to be less than the required power density (E_{TH}) at the relevant value of the spatial distance for a photodisruption with the predetermined probability; and
wherein a difference between the determined power density (Eᵢ) and the required power density (E_{TH}) depends on a product (dₛ×dₜ) of values of the spatial distances dₛ and dₜ or is a linear function or a step function.

9. The laser device according to claim 7;
wherein the control unit controls the deflecting unit such that target volumes with spatial distances dₛ above the limit distance result in an outer annular region of an interaction zone field and target volumes with spatial distances below the limit distance result in an inner region of the interaction zone field.

10. The laser device according to claim 1;
wherein the control unit controls the laser during the emission of the radiation pulses such that an emission frequency of the laser is constant.

11. The laser device according to claim 1;
wherein the spatial distance dₛ between directly successive target volumes is between 0.5 μm and 5 μm.

12. A method for severing eye tissue, comprising:
inputting energy by means of a sequence of radiation pulses focused in target volumes so as to generate a field of interaction zones;
wherein, for each radiation pulse of the sequence of radiation pulses, a power density (Eᵢ) is inputted into a relevant target volume in mathematically monotonically increasing dependency on a spatial distance dₛ between two target volumes of directly successive radiation pulses.

13. The method according to claim 12;
wherein target volumes are located on a track along a curved spatial area;
wherein the power density (Eᵢ) inputted into the relevant target volume additionally depends in a mathematically monotonically increasing manner upon a spatial distance dₜ between two adjacent track portions of two different cycles of the track.

14. A computer-readable storage medium encoded with instructions that when executed by at least one processor within a computer system, that comprises at least one interface operatively coupled to the at least one processor, cause the computer system to interact with a laser device to enable actions comprising the method of claim 12.
